(19) United States
(12) Patent Application Publication
(10) Pub. No.: US 2014/0142605 A1
(43) Pub. Date: May 22, 2014

(54) TISSUE DISSECTOR AND INCISOR

(71) Applicant: Jorge Lopera, San Antonio, TX (US)

(72) Inventor: Jorge Lopera, San Antonio, TX (US)

(73) Assignee: The Board of Regents of the University of Texas System, Austin, TX (US)

(21) Appl. No.: 14/072,950

(22) Filed: Nov. 6, 2013

Related U.S. Application Data
(60) Provisional application No. 61/722,822, filed on Nov. 6, 2012.

(51) Int. Cl. A61B 17/34

(52) U.S. Cl. CPC A61B 17/3417; A61B 17/3496
USPC 606/185

(57) ABSTRACT

The device described herein facilitates interventional medical procedures, reduces the formation of skin tag, and increases the safety of such procedures relative to the operator and patient.

This application is a non-provisional application of U.S. Provisional Patent application Ser. No. 61/722,822 filed Nov. 6, 2012, which is incorporated herein by reference in its entirety.

### BACKGROUND

The use of minimally invasive procedures has grown exponentially in the last years and it is expected to continue to grow. After needle puncture, a surgical blade is used to open the access skin tract followed many times by tissue dissection with forceps to facilitate placement of larger access devices and tubes. This step is time consuming. It also poses additional risks to the operator by the use of a sharp surgical scalpel, and to the patient by potentially too deep tissue penetration by the surgical blade. Very commonly there is inadequate incision of the skin that causes a “skin tag”. The presence of this tag may result in guide wire kinking and damage to the tip of the access devices with potential injury to the patient. The generation of a skin tag usually requires reusing the surgical blade for additional incisions to remedy the situation.

Thus, there remains a need for additional device designs that provide a safer device and to reduce the occurrence of skin tag.

### SUMMARY

Certain embodiments are directed to a lumen access blade and devices comprising the same. The blade can comprise a blade body having a long and short axis, a top, two sides, and a bottom. In certain aspects the blade has a cutting edge at the proximal end of the long axis of the blade body, wherein the distal end of the long axis is configured for attachment to a handle. In certain aspects the handle has a retractable mechanism that moves the blade from a safety position to operative position. In a further aspect the blade forms a groove or lumen through which an implement (guidewire or puncture implement) can be positioned. In certain aspects the groove runs the length of the long axis of the blade body. In other aspects the groove can be tapered into the long axis of body and as such need not run the length of the blade. In certain embodiments the blade is operatively position with respect to a guidewire or puncture implement (e.g., needle or trocar). In a further embodiment the blade body tapers to the cutting edge. The blade can be manufactured out of metal, in certain embodiments stainless steel. In certain embodiments the implement groove bisects the cutting edge of the blade.

Certain embodiments are directed to a surgical instrument for accessing body lumens. In certain aspects the instrument comprises a retractable blade that can be operatively coupled to an puncture implement. The blade can provide for an incision further opening a puncture. A housing can be operatively coupled to the blade, wherein the blade can be placed in an operating position or a safety position with the housing shielding the blade when the blade is in the safety position. The blade can be coupled to a retractable mechanism. The retractable mechanism can be operatively coupled to a mechanism for the extension and retraction of the blade (e.g., switch, push tab or the like). The extension/retraction mechanism can be located on the left side, right side, top, bottom, or distal end of the handle. The blade can be configured to have a groove on the bottom, top, either side, or through the blade so that the blade can be slid along the long axis of the puncture implement.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. An illustration of a lumen access blade.

FIG. 2. An illustration of a lumen access blade from a second perspective.

FIG. 3. An illustration of a lumen access blade taken from a bottom view of the blade.

FIG. 4. An illustration of a lumen access blade incorporated into a surgical instrument.

FIG. 5. An illustration of a lumen access blade incorporated into a surgical instrument.

FIG. 6. An illustration of a surgical instrument in the safety position.

### DESCRIPTION

Thousands of minimally invasive procedures are performed every day based on the standard Seldinger technique. In performing the Seldinger technique the desired vessel or cavity is punctured with a sharp hollow needle called a trocar. A round-tipped guidewire is then advanced through the lumen of the trocar, and the trocar is withdrawn. Typically a “sheath” or blunt cannula is passed over the guidewire into the cavity or vessel. Also, drainage tubes can be passed over the guidewire (as in chest drains or nephrostomies). Once the sheath, cannula, or tube is in position the guidewire is withdrawn. After puncture a small incision is routinely performed with a surgical blade to prevent the skin from damaging the access devices and facilitate placement. Upon completion of the procedure, the sheath is withdrawn. In certain settings, a sealing device may be used to close the hole made by the procedure.

The sheath, cannula, or tube can be used to introduce catheters or other devices to perform endoluminal (inside the hollow organ) procedures, such as angioplasty. Various imaging techniques can be used during the technique, if necessary, e.g., ultrasound. In certain aspects, Fluoroscopy can be used to confirm the position of a catheter and to maneuver it to the desired location. In other aspects, radio-contrast agents can be administered for visualization of organs.

These techniques are used for angiography, insertion of chest drains and central venous catheters, insertion of PEG tubes using the push technique, insertion of leads for an artificial pacemaker or implantable cardioverter-defibrillator, thermoablation, angioplasty, embolization, biopsy, and numerous other interventional medical procedures.

The devices described herein facilitate interventional medical procedures, reduce the formation of skin tag, and increase the safety of such procedures relative to the operator and patient. If an incision is not properly done additional incisions are required to remove a skin tag. The need for repeating such a procedure exposes the operator or patient to accidental injuries with the surgical blade. Furthermore, the risk of injury is increased in obese patients. Problems such as these are encountered in clinical practice on a daily basis. Thus there is a need for a simple and more secure way to cut and dissect tissues during initial access.

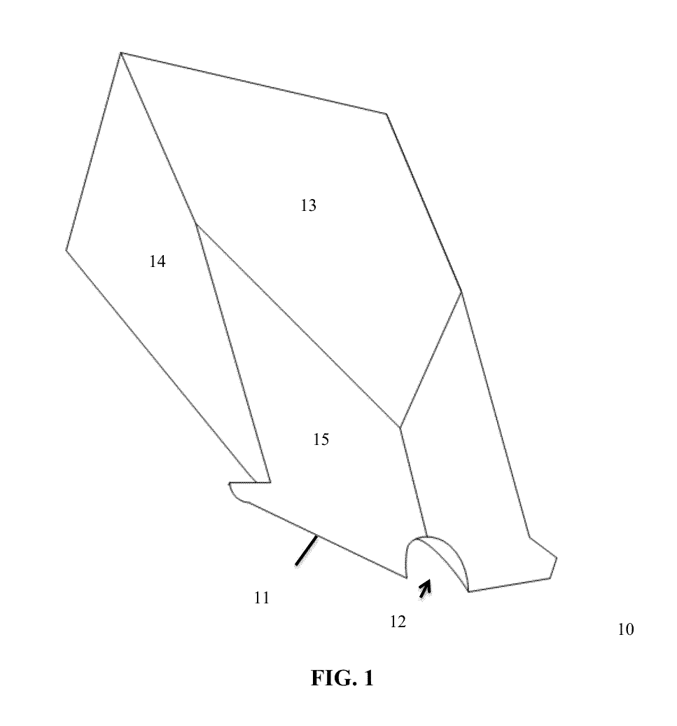

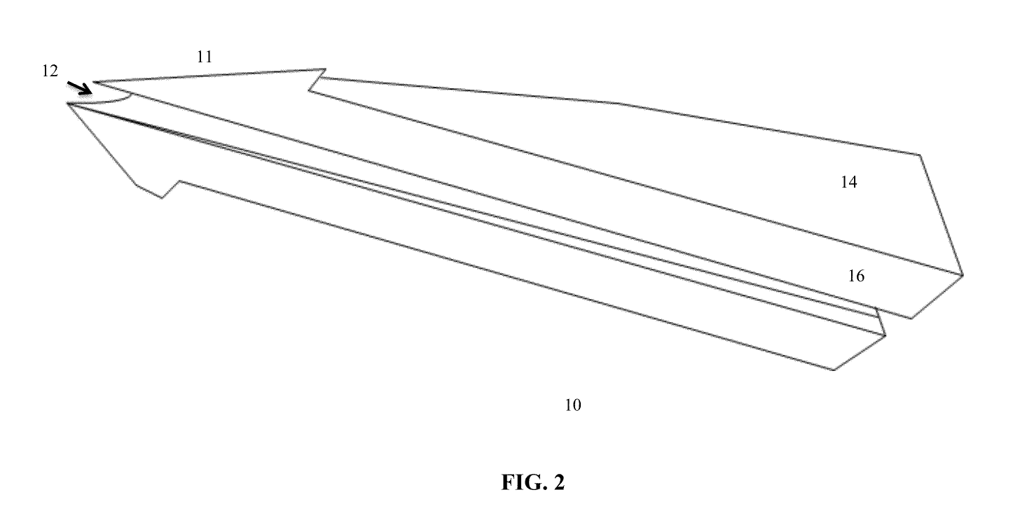

The current device improves efficiency and operator/patient safety during procedures, such as the Seldinger procedure, by providing a device having a retractable cutting blade that is contained with in a protective housing until needed. The blade is configured to integrate with a guidewire or puncture implement. In certain aspects a needle or guidewire is positioned with a groove on the blade and the blade is slid along the needle or guidewire until the appropriate incision is made, this groove decrease/eliminates the chances of leaving the skin tag behind. FIG. 1 illustrates and embodiment of cutting blade 10. The illustration shows a top 13, one of two sidewalls 14, secondary tapers 15, a cutting edge 11, and a groove 12. The body of the blade has a primary taper and in certain aspects a secondary taper 15 to the blade. The blade forms a groove 12 along the long axis of the blade. In certain aspects the groove is located on the bottom of the blade 16 (see FIG. 2). The groove may also be formed on the top or side of the blade or through the blade. In certain aspects the blade comprises a lumen that runs through the blade. The groove is configured to receive a puncture implement, such as a needle, or a guidewire. The groove allows the cutting edge to be placed in the appropriate position for making an incision at the puncture site. The incision provides access for the placement of other instruments and devices without producing a skin tag.

FIG. 2 illustrates another perspective of the blade illustrated in FIG. 1. In FIG. 2 the side wall of the blade body 14 is visible as well as the bottom 16 of the blade. In this particular embodiment groove 12 is positioned in the bottom 16 of the blade. The groove also bisects cutting edge 11.

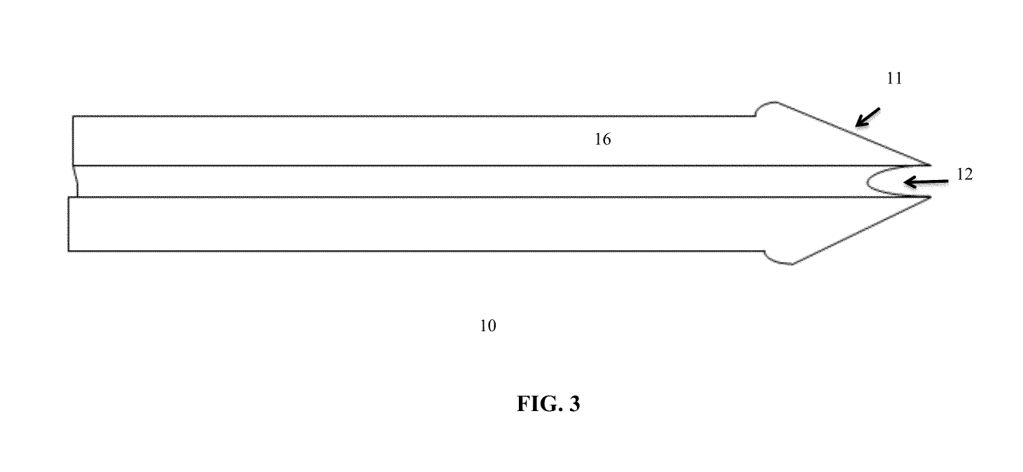

FIG. 3 is a photograph of an embodiment of the blade taken from the groove side showing groove 12 running the length of the long axis of blade 10.

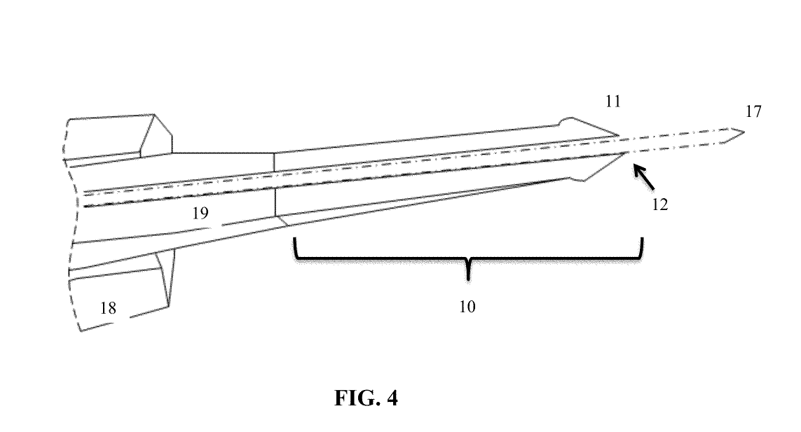

FIG. 4 is a photograph illustrating blade 10 operatively coupled to handle 18 and moveable mechanism 19. Blade 10 is shown with needle 17 positioned in groove 12 with cutting edge 11 flanking needle 17.

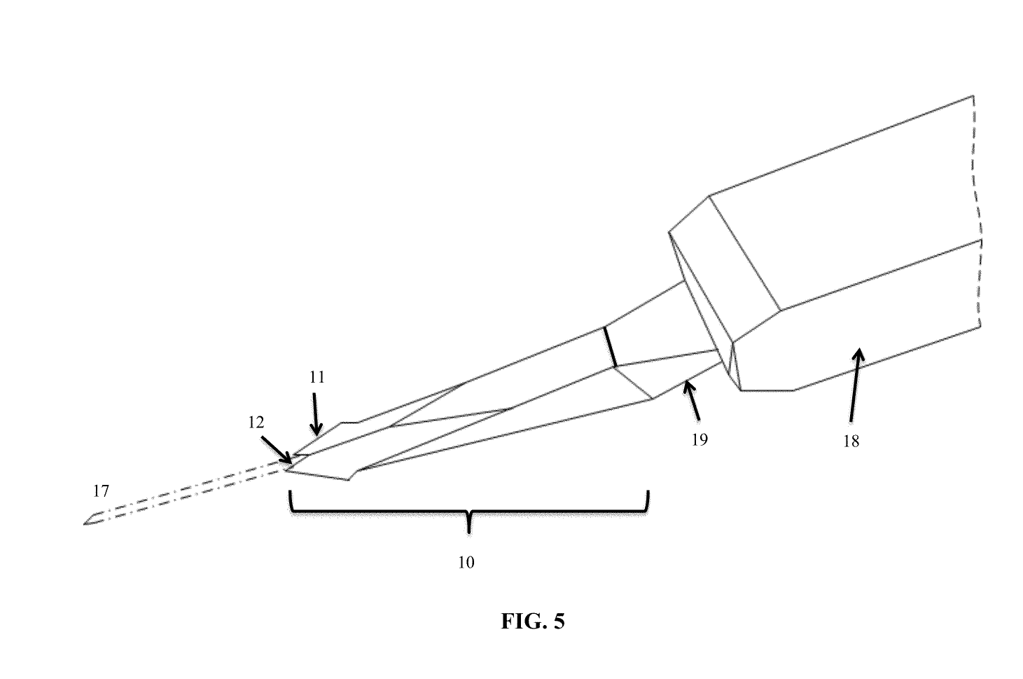

FIG. 5 is a photograph of blade 10 in an operative position in relation to handle 18 by use of moveable mechanism 19. The photograph is a top view of blade 10 being used with needle 17. Blade 10 is positioned on needle 17 where it is moved into position to make an incision in the area adjacent to a puncture. In other aspects blade 10 can be position with the aid of groove 12 relative to a guidewire that has been placed using needle 17.

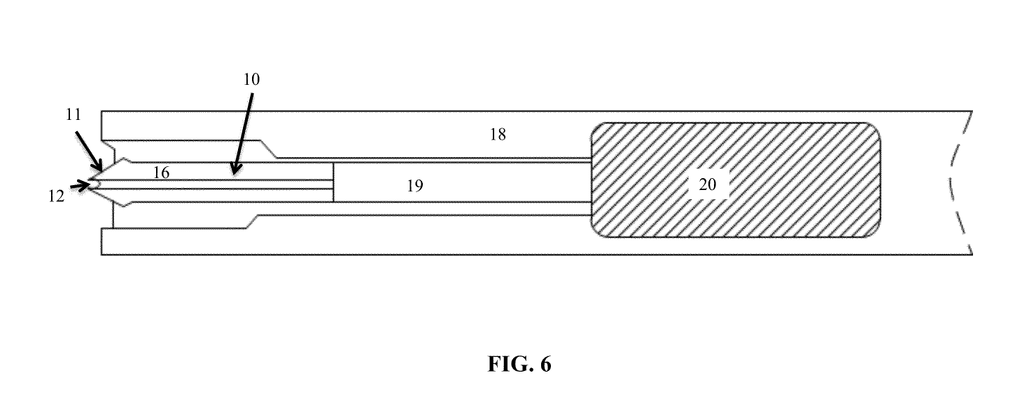

FIG. 6 is a photograph of blade 10 in a retracted or safety position in relation to handle 18. Moveable mechanism 19 has been retracted in to a sheath or protective cover 20.

Certain embodiments allow the operator to expose a special surgical blade that will cut tissue with ease, without exposing the operator to the cutting blade unnecessarily. The design also reduces or eliminates the risk of leaving a skin tag behind. The device can be configured for use in conjunction with a puncture implement (e.g., a needle) or a guidewire with a groove in the blade facilitating the localization of the cutting edge. A needle can be any gauge needle, e.g., 21 to 18 gauge (G) needle.

A device can consist of a handle 18 that allows the operator to expose a surgical blade 10 that will cut the tissue to the desired depth; the blade 10 will then retract in to handle 18 to avoid accidental exposure to the operator or patient. A blade 10 can be positioned over a needle, groove 12 at the bottom of blade 10 allows it to slide over needle 17, once the device is in close proximity to the skin, and blade 10 is advanced to the desired depth by advancing forward the moveable mechanism 19 of the device. Once the incision is created blade 10 is retracted and the device removed. In certain aspects blade 10 can be configured to allow simultaneous tissue dissection, e.g, blade 10 can be tapered so that the skin is manipulated during or after the incision.

The device described herein can be used to initiate or during minimally invasive procedures. The device can be used when a surgical blade is needed or used to open the access skin tract and for tissue dissection to facilitate placement of larger access devices and tubes.

1. An lumen access blade comprising:
(a) a blade body having a long and short axis;
(b) a cutting edge at the proximal end of the long axis of the blade body, wherein the distal end of the long axis is configured for attachment to a handle; and
(c) a implement groove or lumen running the length of the long axis of the blade body to provide for insertion of a guidewire or puncture implement.

2. The blade of claim 1, wherein the blade body tapers to the cutting edge.

3. The blade of claim 1, wherein the blade is a metal blade.

4. The blade of claim 3, wherein the blade is a surgical stainless steel blade.

5. The blade of claim 1, wherein the implement groove bisects the cutting edge.

7. A surgical instrument for accessing body lumens comprising:
(a) a retractable blade that can be operatively coupled to an implement, wherein the blade provides an incision further opening the puncture;
(b) a housing operatively coupled to the blade, wherein the blade can be placed in an operating position or a safety position.

8. The surgical instrument of claim 7, wherein the blade is configured to have a groove on the bottom of the blade so that the blade can be slid along the long axis of the puncture implement.
